(19) United States
(12) Patent Application Publication
(10) Pub. No.: US 2002/0082484 A1
(43) Pub. Date: Jun. 27, 2002

(54) Image display control system and method

(76) Inventors: Akiko Baba, Amagasaki-shi (JP); Norihiro Akasaka, Amagasaki-shi (JP); Osamu Ozaki, Amagasaki-shi (JP)

Correspondence Address: WENDEROTH, LIND & PONACK, L.L.P., 2033 K STREET N. W., SUITE 800, WASHINGTON, DC, 20006-1021, US

(21) Appl. No.: 09/998,177

(22) Filed: Dec. 3, 2001

(30) Foreign Application Priority Data
Dec. 27, 2000 (JP) 2000-398814

(51) Int. Cl. A61B 5/00

(52) U.S. Cl. 600/300

(57) ABSTRACT

In an image display control system having a display unit, a plurality of first thumbnail images of a plurality of radiographic images and a plurality of second thumbnail images of a plurality of temporally processed images are displayed by a displaying unit on a screen of the display unit while the first and second thumbnail images are tabulated to form a matrix image. The radiographic images are taken at different points of time, the temporally processed images are produced by combining the radiographic images. A plurality of the thumbnail images on the matrix image is selected by a selecting unit so that a plurality of images are sequentially displayed by sequentially displaying unit. The plurality of images corresponds to the selected thumbnail images.

### BACKGROUND OF THE INVENTION

1. Field of the Invention

The present invention relates to image display control system and method for scaling down a plurality of temporally processed images and a plurality of radiographic images that are original images of the temporally processed images, thus displaying the down-scaled images while they are tabulated in a matrix, and, in particular, relates to image display control system and method for selecting a plurality of images so as to sequentially diagnose and read the selected images.

2. Description of the Related Art

A conventional image display control system for displaying the down-scaled images while they are tabulated in a matrix is disclosed in Japanese Patent Publication No. 10-155746.

That is, the conventional image display control system displays, on a screen of a display unit, first thumbnail images in a plurality of radiographic images of a patient that are taken at different points of time, respectively, and second thumbnail images in a plurality of temporally processed images each of which is produced by combining at least two arbitrary images in the plurality of radiographic images, while the first and second thumbnail images are tabulated to form a matrix. The tabulated first and second thumbnail images clearly show correspondences between the radiographic images and the temporally processed images, thus making it possible to easily recognize a history of taken images, a diagnosis history and a reading history of the patient.

However, in the conventional image display control system, it is not permitted to simultaneously select arbitrary two images in the tabulated matrix image, making it impossible to sequentially diagnose and read the plurality of radiographic images or temporally processed images. Therefore, in cases of reading another arbitrary image in the tabulated matrix image, after reading one of the radiographic images (or the temporally processed images) on the screen, it must be necessary to display the tabulated matrix image again so as to select one thumbnail image in the tabulated matrix image, that a user such as, a radiologist, a diagnostician or the like (referred to simply user hereinafter) want to read, thereby deteriorating the operationality of the image display control system.

In the conventional image display control system, when selecting one thumbnail temporally processed image in the tabulated matrix image, the selected thumbnail temporally processed image is only scaled up to be displayed on the screen. When the user intends to display the original radiographic images corresponding to the selected thumbnail temporally processed image, it must be necessary for the user to execute a troublesome operation, such as an operation of clicking another button of “full size image display”.

In the conventional image display control system, in cases where a temporally processed image corresponding to the selected thumbnail temporally processed image is not produced, when selecting the thumbnail temporally processed image in the tabulated matrix image, a message of inquiring whether or not to execute to produce the temporally processed image corresponding to the selected thumbnail image is displayed by the system on the screen.

The user clicks “execute” so that the system executes to produce the temporally processed image, thereby requiring a lot of time until the user diagnoses and reads the temporally processed image corresponding to the selected thumbnail image.

In the conventional image display control system, because it is not to set to protect each image in the tabulated matrix image, there is the possibility of accidentally deleting at least one of the radiographic images and the temporally processed images.

Because the user does not confirm, on the tabulated matrix image, that whether or not each of the radiographic images and the temporally processed images is already diagnosed, there is the possibility of repeatedly diagnosing the same image.

In cases where the screen size of the display unit to be used, the size of the diagnosis image area and each size of each window element on the image do not coincide with each other when diagnosing and reading the image, malfunction such that characters on each of the window elements are small and so on occurs, thereby deteriorating the operationality of the image display control system.

In addition, when selecting one of the patients in the patient list, the system reads a number of images of the selected patient so as to display them. However, in cases where it is necessary to read images of the plurality of patients, it must be required for the user to specially select the plurality of patients so as to wait until all of the images of the selected patients are read, thereby requiring a lot of time to search one of the images or obtaining data.

### SUMMARY OF THE INVENTION

This invention is directed to overcome the foregoing problems in the conventional art.

Accordingly, it is an object of the present invention to provide an image display control system and method capable of, when a user intends to diagnose and read a plurality of images, previously selecting the plurality of images which are intended to be diagnosed and read, thereby sequentially diagnosing the selected images without requiring additional time to select the images.

In order to achieve such object, according to one aspect of the present invention, there is provided an image display control system having a display unit, said image display control system comprising:

means for displaying a plurality of first thumbnail images of a plurality of radiographic images and a plurality of second thumbnail images of a plurality of temporally processed images on a screen of the display unit while the first and second thumbnail images are tabulated to form a matrix image,

the radiographic images being taken at different points of time, said temporally processed images being produced by combining the radiographic images;

means for selecting a plurality of the thumbnail images on the matrix image; and

means for sequentially displaying a plurality of images, said plurality of images corresponding to the selected thumbnail images.

Then, the term “radiographic image” used herein is employed to mean “digitized image which is produced by digitizing a radiographic obtained by transmitting a radiation through a human body” such as a front chest radiograph, a side chest radiograph and so on.

In addition, the term “temporally processed image” used herein is employed to mean “image obtained by performing image processing with using two radiographic images” such as a temporal subtraction image, energy subtraction image and so on.

Furthermore, the term “temporal subtraction image” used herein is employed to mean “subtraction image produced by processing two radiographic images which are taken at different points of time with the use of algorithm such as “temporal subtraction technique”. On the temporal subtraction image, it is possible to enhance an interval change portion occurring in the two radiographic images. Incidentally, the technique of producing the temporal subtraction image is disclosed in Japanese Patent Publication No. 7-37074.

Still furthermore, the term “energy subtraction image” used herein is employed to mean “subtraction image between two radiographic images which are obtained by using different voltages of an X-ray tube. By obtaining the difference (difference absorbability of X-ray) between the two radiographic images which are obtained by using different voltages of the X-ray tube, it is possible to enhance conditions of each of the tissues of the human body, including bones, vessels and the like.

In addition, as a display unit, a CRT (Cathode-ray Tube), a plasma display, a liquid crystal display or the like can be utilized. Because the present invention relates to a medical system, it is preferable that the display unit having high resolution, such as 1000 scanning lines class or more is used, because this system is a medical system.

According to one aspect of the present invention, in cases where the user intends to diagnose plural images, it is possible to previously select the plural images by the image selecting means, so as to sequentially diagnose the plural images without requiring additional time to select the plural images, thus reducing operational time when diagnosing and reading the plural images.

The preferred embodiment of this aspect has an arrangement that the sequentially displaying means displays, when the second thumbnail images are selected by the selecting means, the radiographic images which are original images of the temporally processed images corresponding to the selected thumbnail images.

According to this preferred embodiment of this one aspect, because the sequentially displaying means displays, when the second thumbnail images are selected by the selecting means, the radiographic images which are original images of the temporally processed images corresponding to the selected thumbnail images, there is no need of specially operations for displaying the temporally processed image, so that it is possible to improve the operability.

The preferred embodiment of this aspect has an arrangement that the sequentially displaying means displays the plurality of images in an order, and the order is permitted to be freely set.

Because the order is permitted to be freely set, it is possible to sequentially display the images from the image that the user extremely intends to diagnose, thereby efficiently executing the diagnosis of the images.

The preferred embodiment of this aspect has an arrangement of comprising, when an area on the matrix image which corresponds to a temporally processed image that is not produced yet, is selected, means for automatically producing the temporally processed image corresponding to the selected area.

According to the preferred embodiment of this aspect, it is impossible to produce the temporally processed image again, thus making short the diagnosis time.

The preferred embodiment of this aspect has an arrangement of comprising means for setting protection information to at least one of the first and second thumbnail images on the matrix image, the protection information indicating that the image of the thumbnail image to which the protection information is set is prevented from being deleted, the protection information setting means being permitted to release the set protection information on the matrix image.

According to the preferred embodiment of this one aspect, it is possible to prevent the user from deleting the image by mistake.

The preferred embodiment of this aspect has an arrangement that a state such that whether the protection information is set to the thumbnail image or released therefrom is permitted to be confirmed on the matrix image.

According to the preferred embodiment of this one aspect, it is possible for the user to easily confirm whether or not the protect information is set.

The preferred embodiment of this aspect has an arrangement of comprising means for setting diagnosis completion information to at least one of the radiographic image and the temporally processed image, the diagnosis completion information indicating that a diagnosis of the corresponding image is already completed.

According to the preferred embodiment of this one aspect, it is possible to prevent the user from repeatedly diagnosing the same images, thus reducing the cost for diagnosis work.

The preferred embodiment of this aspect has an arrangement of comprising means for automatically adjusting a size of an window element according to a resolution of the screen, the window element being displayed on the screen.

According to the preferred embodiment of this one aspect, it is possible to automatically adjust each size of each window element so as to set it to suitable size of the used display unit, thereby displaying each window element on the screen with its size suitably adjusted.

The preferred embodiment of this aspect has an arrangement of comprising means for automatically adjusting a layout in the screen according to an aspect ratio of the display unit so that it is possible to automatically set the suitable layout in the screen according to the display unit, thereby diagnosing the images on the screen on which the image area and the window element are suitably arranged.

The preferred embodiment of this aspect has an arrangement of comprising means for storing thereon the temporally processed images and the radiographic images which are original images thereof while the temporally processed images and the radiographic images are related to each other, so that it is possible to decrease the searching time of the images and the obtaining time thereof.

In order to achieve such object, according to another aspect of the present invention, there is provided an image display control system comprising:

means for storing thereon a plurality of items of information of patients, a plurality of radiographic images corresponding to the patients and a plurality of temporally processed images corresponding thereto;

means for displaying a list of the items of information of the patients so as to simultaneously select the items of information of the patients which are intended for diagnosis on the list; and

means for combining the stored temporally processed images and the radiographic images which are original images thereof, in correspondence with the selected patients, thus sequentially displaying the combined images.

According to another aspect of the present invention, when reading plural images of the patients, it is possible for the user to prevent the user from selecting each patient and waiting until all images are read, thereby omitting time required for the searching operation and data obtaining operation.

The preferred embodiment of this another aspect has an arrangement that the sequentially displaying means displays the combined images in an order, and the order is permitted to be freely set so that it is possible to sequentially display the images from the image that the user extremely intends to diagnose, thereby efficiently executing the diagnosis of the images.

In order to achieve such object, according to further aspect of the present invention, there is provided an image display control method comprising the steps of:

preparing a display unit;

displaying a plurality of first thumbnail images of a plurality of radiographic images and a plurality of second thumbnail images of a plurality of temporally processed images on a screen of a display unit while the first and second thumbnail images are tabulated to form a matrix image,

the radiographic images being taken at different points of time, the temporally processed images being produced by combining the radiographic images;

selecting a plurality of the thumbnail images on the matrix image; and

sequentially displaying a plurality of images, the plurality of images corresponding to the selected thumbnail images.

In order to achieve such object, according to still further aspect of the present invention, there is provided an image display control method comprising the steps of:

storing on a data storage unit a plurality of items of information of patients, a plurality of radiographic images corresponding to the patients and a plurality of temporally processed images corresponding thereto;

displaying a list of the items of information of the patients so as to simultaneously select the items of information of the patients which are intended for diagnosis on the list; and

combining the stored temporally processed images and the radiographic images which are original images thereof, in correspondence with the selected patients, thus sequentially displaying the combined images.

### BRIEF DESCRIPTION OF THE DRAWINGS

Other objects and aspects of the present invention will become apparent from the following description of an embodiment with reference to the accompanying drawings in which:

FIG. 1 is a block diagram showing an overall configuration of an image display control system according to an embodiment of the present invention;

FIG. 2 is a view showing a diagnosis object selecting image including a matrix image of the image display control system shown in FIG. 1;

FIG. 3A is a view showing only a matrix image area of the matrix image shown in FIG. 2;

FIG. 3B is a view showing an order of displaying images when diagnosing the images according to FIG. 3A;

FIG. 4A is a view showing a case of having two CRTs shown in FIG. 1, which show diagnosis images;

FIG. 4B is a view showing a case of having three CRTs shown in FIG. 1, which show diagnosis images;

FIG. 5 is a view showing an example of control panel for changing over images according to the embodiment of the present invention;

FIG. 6A is a view showing a layout example of the diagnosis image according to the embodiment of the present invention;

FIG. 6B is a view showing a layout example of the diagnosis image displayed on a screen having different resolution as compared with that of FIG. 6A;

FIG. 7 is a view showing a a layout example of the diagnosis image displayed on a screen whose vertical side is longer than its horizontal side; and

FIG. 8 is a view showing an example of a tabulated image having a patient list area.

### DESCRIPTION OF THE PREFERRED EMBODIMENT

A preferred embodiment of the present invention will be described hereinafter with reference to the accompanying drawings.

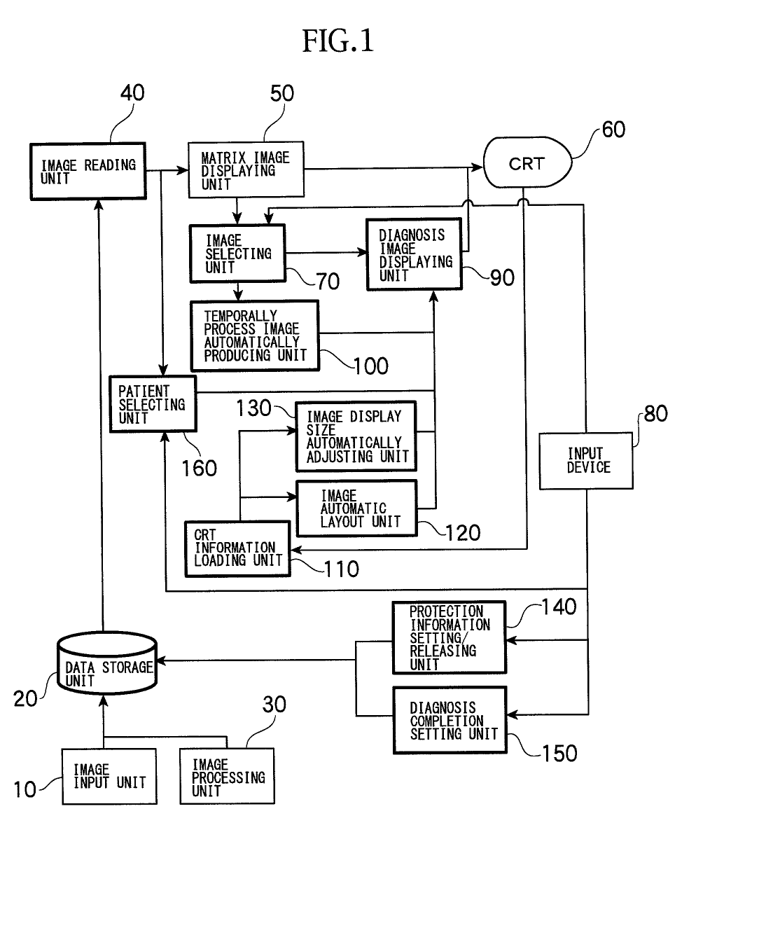

FIG. 1 is a block diagram showing an overall configuration of an image display control system according to an embodiment of the present invention

An image input unit 10 is operative to digitize a taken radiograph. The digitized radiograph is stored as a radiographic image in a data storage unit 20 of this system.

In case of digitizing a radiograph, this unit 10 includes a device for digitizing a film such as a film digitizer or the like, and in case of digitally taking a radiograph, the unit 10 includes a digital imaging unit, such as a CR (computed radiography) unit. Any device or unit, which is capable of producing radiographic image can be used as the image input unit 10.

The data storage unit 20 stores not only radiographic images inputted from the image input unit 10 but also temporally processed images generated on the basis of the radiographic images. The data storage unit 20 also stores each patient ID (identification) as patient identifying information corresponding to each radiographic image, each image ID as image identifying information corresponding to each radiographic image and each temporally processed image, each taking time (date and time) corresponding to each radiographic image, and protection information of each radiographic image and each temporally processed image.

In this embodiment, as the data storage unit 20, a database is used. The data storage unit (database) 20 collectively manages patient information of the patients, radiographic images of the patients, information (data) indicating correspondences between original images (radiographic images) and temporally processed images and so on, thus reducing processing time required for searching and collecting desired image or desired information (data).

In particular, because the temporally processed images and the radiographic images as the original images thereof are related to each other in the database, when selecting one of the temporally processed images, the system simultaneously collects the original images corresponding to the selected temporally processed image from the database so as to display the collected original images.

In addition, because the patient information of each patient and images (radiographic images and temporally processed images) that are belong to each patient are related to each other, the system displays the patients (patient IDs), while they are tabulated and, when selecting a plurality of patients on the tabulated patient's image, and combines the temporally processed images stored on the database 20 in correspondence with each of the patients to original images (radiographic images) of the respective temporally processed images, thus sequentially displaying the combined images.

When storing the patient IDs and the images on the database as the data storage unit 20, it is possible to easily collect data (image or information) from the database. In addition, because of easily searching data according to searching keys, such as patient IDs, image IDs and so on, thereby reducing the searching time as compared with using files as the data storage unit 20.

When acquiring data searched with the searching key, the database as the data storage unit 20 makes short the acquiring processing. Incidentally, each patient ID and each image ID are unique.

An image processing unit 30 produces temporally processed images on the basis of the radiographic images stored on the data storage unit 20, and maintenances each temporally processed image and each radiographic image.

An image reading unit 40 reads images for tabulated matrix image or diagnosis image from the temporally processed images and the radiographic images each two images of which are original images of each of the temporally processed images, and reads image information (information including at least one of the patient IDs corresponding to the read images, the image IDs corresponding thereto or the like.

A matrix image displaying unit 50 displays the read images and the read information while the read images and the read information are tabulated to form a matrix.

That is, the matrix image displaying unit 50 displays on a screen of a CRT, first thumbnail images constituting radiographic images that are taken at different points of time and second thumbnail images constituting temporally processed images each of which is produced by combining two arbitrary images in the radiographic images, while the first and second thumbnail images are tabulated to form a matrix.

An image selecting unit 70, as image selecting means in the present invention, selects at least one of images corresponding to the displayed thumbnail images according to the specify of at least one of the thumbnail images with an input device 80. The thumbnail images on the tabulated matrix image, that a user can specify with the input device 80, corresponds to the temporally processed images and the radiographic images which are the original images thereof so that, when executing diagnosis and reading, it is possible for the user to select simultaneously the plurality of thumbnail images.

The input device 80 transmits commands for selecting at least one of the thumbnail images corresponding to the temporally processed images and the radiographic images, setting protection information and canceling the set protection information according to the user's operation of the input device 80. A mouse is generally and reasonably used as this device, however a touch pen, a keyboard or the similar devices can be substituted.

The images corresponding to the selected thumbnail images are overlapped on the diagnosis image displayed on the CRT 60 and produced by the diagnosis image displaying unit 90 so that the user sequentially reads and diagnoses the displayed images.

Then, the image display processing at the time of diagnosis is explained in detail.

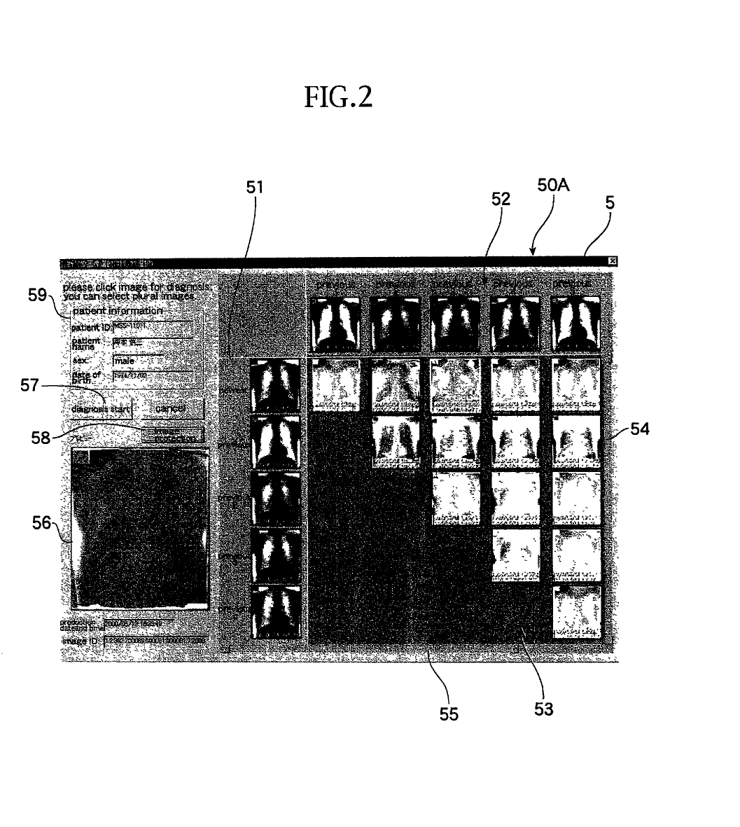

FIG. 2 shows an image (diagnosis object selecting image) for selecting a diagnosis object (matrix select) including the produced matrix image by the matrix image displaying unit 50. On a matrix image area 50A in the diagnosis object selecting image, the radiographic images of one of the patients, which are stored on the data storage unit 20 and the temporally processed images which are produced by combining the radiographic images are displayed as the thumbnail images while they are tabulated to form a matrix.

Window elements laid out on the matrix image area (thumbnail image area) 50A include original image areas 51 and 52, a temporally process image area 53, vertical scroll bar 54 and a horizontal scroll bar 55.

An image preview area 56, a diagnosis starting button 57, an image protecting button 58 and patient information 59 including patient name, patient ID and so on are arranged adjacent to the matrix image area 50A on the diagnosis object selecting image.

The thumbnail images of the radiographic images of the patient, which are stored on the image storage unit 20, are vertically arranged in arbitrary order (in this embodiment, in sequential) on the original image area 51. That is, the thumbnail images from the newest radiographic image to the oldest radiographic image are sequentially displayed from top of the original image area 51 to bottom thereof. In FIG. 2, the five thumbnail images corresponding to the newest radiographic image and four previous images (PREVIOUS 4˜NEWEST).

The thumbnail images of the radiographic images of the patient, which are stored on the image storage unit 20, are horizontally arranged in arbitrary order (in this embodiment, in sequential) on the original image area 52. That is, the thumbnail images from the radiographic image previous to the newest radiographic image to the oldest radiographic image are sequentially displayed from left of the original image area 52 to right thereof. In FIG. 2, the five thumbnail images corresponding to the five previous images (PREVIOUS 1˜PREVIOUS 5).

The layouts of the original image areas 51 and 52 can be customized according to the command from the user (radiologist or the like), and, the layouts of the original image areas 51 and 52 are previously determined in the system.

The thumbnail images of the temporally processed images are displayed on the temporally process image area 53. The temporally processed image is produced from one radiographic image corresponding to the selected thumbnail image in the original image area 51 and one radiographic image corresponding to the selected thumbnail image in the original image area 52. The display position of the thumbnail image of the temporally processed image correspond to the row position of the selected thumbnail image in the original image area 51 and the column position of the selected thumbnail image in the original image area 52.

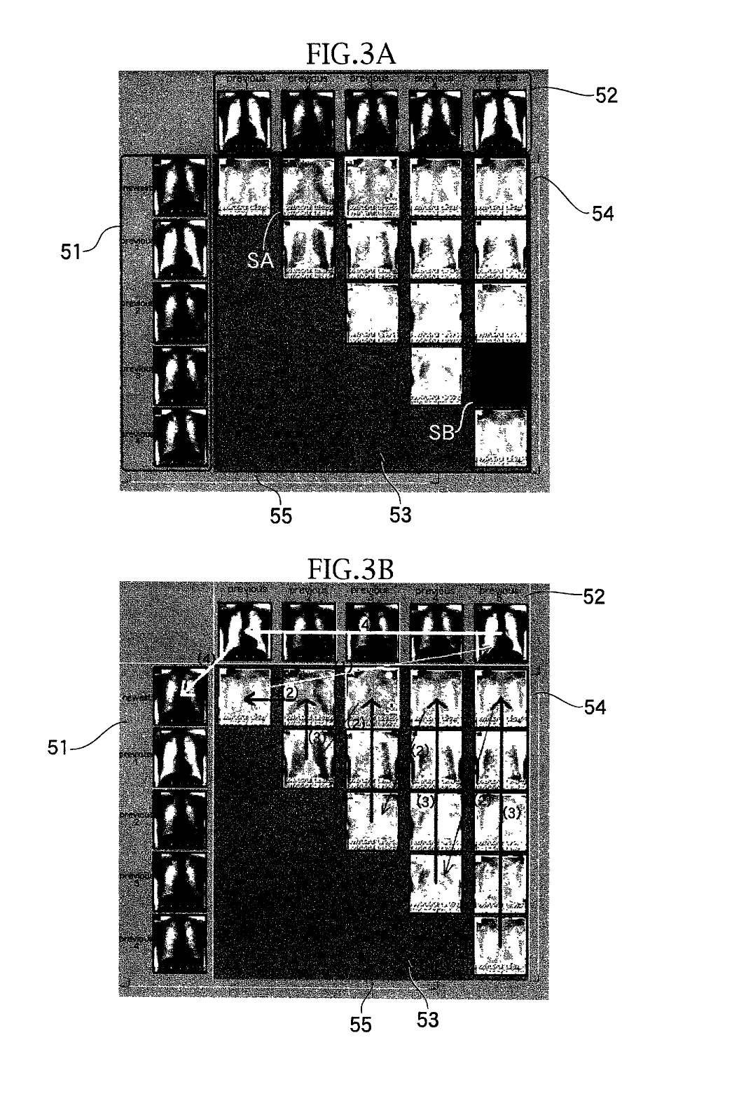

For example, as shown in FIG. 3A, the thumbnail image produced on the basis of the newest original image and the original image of the previous 2 is displayed on an image area SA in the temporally process image area 53. On an image area SB, because the temporally processed image corresponding to the image area SB is not produced yet, no image is displayed.

The vertical and horizontal scroll bars 54 and 55 are used in cases where a plurality of radiographic images of the selected patient are stored on the data storage unit 20 so that all images of the selected patient are not displayed on the screen of the CRT 60. The user operates the vertical and horizontal scroll bars 54 and 55 so that the matrix image displaying unit 50 scrolls images across the screen of the CRT 60, thus displaying a target image on the screen thereof.

Because of selecting arbitrary at least one pair of the original images in the original images, it is possible to produce temporally processed images on the basis of variety combinations of the original images, thus diagnosing the produced temporally processed images.

As described above, the original image areas 51, 52 are arranged on the first column and the first row in the matrix image area 50A so that the original images are sequentially displayed on the original image areas 51, 52. The temporally process image area 53 is arranged on and after the second column and the second row so that the temporally processed images corresponding to the respective original images are displayed on the temporally process image area 53, thus making clear the correspondence between the original images and the temporally processed images. The display positions of the original images and the temporally processed images are not limited to the above positions. For example, the original images are sequentially displayed on the last column and last low in the matrix image area 50A, and the temporally processed images are displayed on another rows and columns except for the first row and first column in the matrix image area 50A.

The preview area 56 is laid out on the screen of the CRT 60 so as to be separated from the matrix image area 50A. The selected one of the thumbnail images corresponding to temporally processed images and the original images displayed on the matrix image area 50A is previewed on the preview with its image size large.

It is possible for the user to select with the input device 80 the temporally processed images and the radiographic images on the matrix image area 50A. For example, the input device 80 is a mouse, when the user operates the mouse to click at least one of the thumbnail images on the matrix image area 50A, thus selecting the at least one of the thumbnail images.

The image selecting unit 70, in response to the selecting operation, selects at least one of the images corresponding to the selected at least one of the thumbnail images from the plurality of radiographic images and temporally processed images read by the image reading unit 40 from the data storage unit 20. It is possible to select simultaneously one, two or more images (radiographic images, temporally processed images).

After selecting at least one of the thumbnail images, when the user pushes down the diagnosis starting button 57 or another button corresponding to the diagnosis, a diagnosis image display unit 90 produces a diagnosis image.

That is, the diagnosis image displaying unit 90 displays on the screen of the CRT 60, at least one image of the radiographic images and the temporally processed images, which corresponds to the selected at least one of the thumbnail images. When the thumbnail image corresponding to the temporally processed image is selected, the diagnosis image displaying unit 90 displays the selected temporally processed image and a pair of original images by which the selected temporally processed image is produced on the diagnosis image. Assuming that each diagnosis image corresponds to each of the selected images, total three images are displayed on one or more CRTs 60.

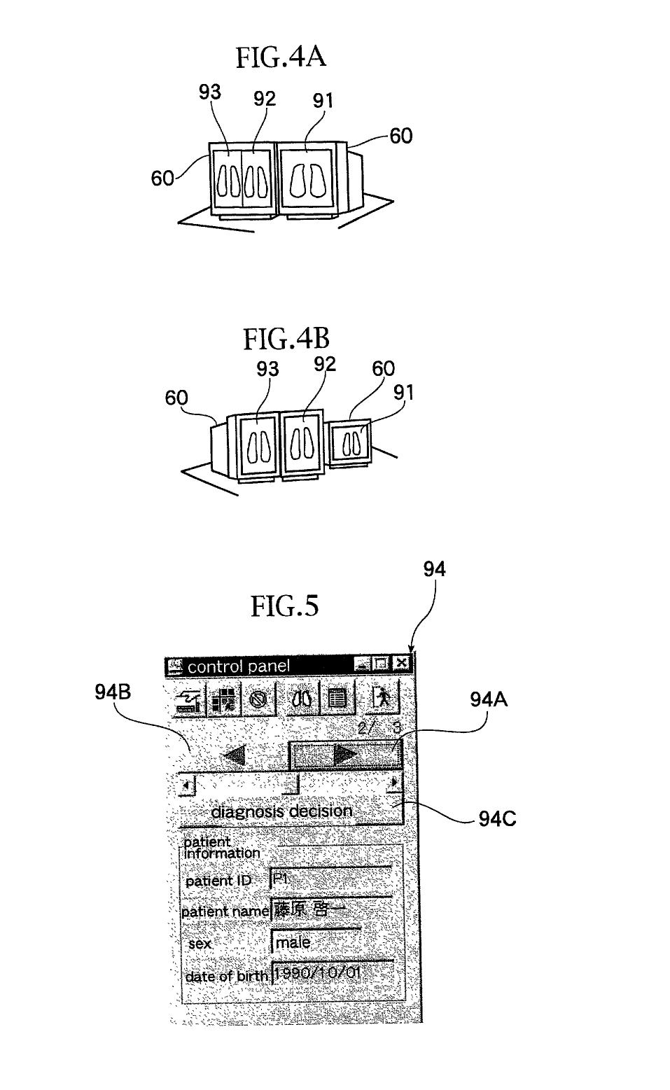

FIG. 4A shows two CRTs 60 one of which displays a diagnosis image 91 for temporally processed images and other of which displays a pair of diagnosis images 92, 93 for radiographic images. FIG. 4B shows three CRTs 60 displaying a diagnosis image 91 for temporally processed images, paired diagnosis images 92, 93 for radiographic images, respectively. Incidentally, when the thumbnail image corresponding to the radiographic image is selected, the diagnosis image displaying unit 90 displays the diagnosis image 92 on one of the CRTs 60.

A control panel (control panel window) 94 shown in FIG. 5 is displayed on the diagnosis image 91 for temporally processed images.

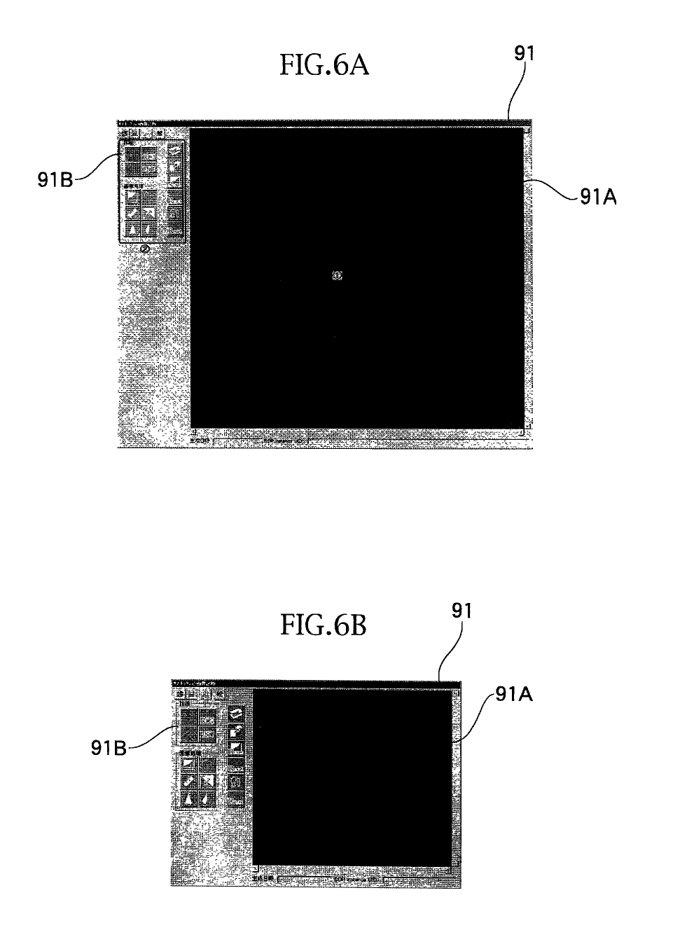

FIG. 6A shows a configuration example of the diagnosis image 91 for temporally processed images.

Window elements laid out on the diagnosis image 91 constitute an image area 91A and an operation button area 91B. The image area 91A is an image area for displaying each temporally processed image, and the operation button area 91B is an area in which operation buttons are collectively indicated.

When selecting the thumbnail image corresponding to the original image, the diagnosis image displaying unit 90 displays the original image corresponding to the selected thumbnail image on the diagnosis image 91, and, on each of the diagnosis images 92 and 93, no image is displayed. When selecting the thumbnail image corresponding to the temporally processed image, the diagnosis image displaying unit 90 displays paired original images corresponding to the selected temporally processed image on the diagnosis images 91 and 92, and displays the temporally processed image on the diagnosis image 93.

The combinations of the paired original images and the temporally processed image to the diagnosis images 91, 92 and 93 are not limited to the above combinations, and can be customized according to the command from the user (radiologist or the like). The information indicating the customized combinations is previously determined in the system.

When diagnosing next image, the user pushes down a right allow button 94A in the control panel 94, or another button corresponding to a change of image so that the diagnosis image display unit 90 changes over from the displayed images on the diagnosis images 91, 92 and 93 to new images, thereby displaying them thereon.

Next, an order of displaying images at the time of diagnosis is explained.

When selecting plural thumbnail images on the matrix image area, it is possible to display the images corresponding to the selected thumbnail images in the order previously determined by the system so that the user (diagnostician) can diagnose in the order the sequentially displayed plural images.

An example in that the plural images are displayed in the order described hereinafter on the basis of the temporally ascending sequence is shown in FIG. 3B.

Numbers in FIG. 3B indicate to display the plural images in accordance with the following rules (1)˜(4):

rule (1) of displaying, when the plural images include the temporally processed image and corresponding original images, firstly the temporally processed image and secondly corresponding original images;

rule (2) of, when the plural images are the temporally processed images, displaying the temporally processed images in order of increasing elapse time from the taking time of each of the older original images on which each of the temporally processed images is produced;

rule (3) of, in the rule (2), when at least two of the older original images have the same elapse time, displaying the temporally processed images using the same older original images in order of increasing elapse time from the taking time of each of the remained original images on which each of the temporally processed images is produced; and

rule (4) of displaying, when the plural images are the original images, displaying the original images in order of increasing elapse times from the taking times of the remained original images.

Incidentally, it is possible to set the display order of the plural images by the diagnosis image displaying unit 90.

Next, returning to FIG. 1, a temporally process image automatically producing unit 100 corresponding to automatically producing means in the present invention is explained hereinafter.

The temporally process image automatically producing unit 100 (hereinafter, referred to simply automatically producing unit 100), in cases where a temporally processed image corresponding to the selected thumbnail image is not produced yet, automatically produces the temporally processed image when starting diagnosis.

For example, no image is displayed on the image area SB because the temporally processed image corresponding to the image area SB is not produced yet, but the user can select the image area SB with the input device 80. After selecting the image area SB, the user pushes down the diagnosis starting button 57, or another button corresponding to the diagnosis so that the automatically producing unit 100 automatically produces the temporally processed image corresponding to the pushed image area SB on the basis of the pair of original images corresponding to the pushed image area SB, whereby the diagnosis image displaying unit 90 displays the produced temporally processed image.

A CRT information loading unit 110 is operative to load information including the image size of the CRT 60 to be used, the resolution thereof and so on. The diagnosis image displaying unit 90 decides the size of the image to be produced, the size of the screen and the display position of the image on the screen according to the loaded information by the CRT information loading unit 110.

When displaying the image, the diagnosis image displaying unit 90 also determines the size of the image, that of the screen and the display position of the image on the screen on the basis of the loaded information of the CRT 60.

Next, an image display size automatically adjusting unit 130 is explained hereinafter.

The image display size automatically adjusting unit 130 (hereinafter, referred to simply adjusting unit 130) corresponding to automatically adjusting means automatically determines each size of each window element on the screen according to the loaded information including the resolution of the CRT 60, and automatically adjusts each size of each window element so as to set it to most suitable size, thereby displaying each window element on the screen with its size suitably adjusted.

For example, when the screen has high resolution, the adjusting unit 130 automatically adjusts the size of the image area to be large according to the high resolution of the screen so as to automatically change the sized of the images displayed on the image area according to the resolution of the screen (the size of the image area).

FIG. 6A shows the screen with the resolution of 1280×1024 pixels, and FIG. 6B shows the screen with the resolution of 800×600 pixels. The image area 91A with respect to the screen in FIG. 6B is smaller than that with respect to the screen in FIG. 6A, but the size of the button area 91B keeps unchanged.

The change of the resolution of the screen does not cause the sizes of the operation buttons to be changed, making it possible to prevent the bad influence of the diagnosis due to the operationality with the operation buttons.

Next, an image automatic layout unit 120 is explained.

The image automatic layout unit 120 corresponding to automatically adjusting means automatically determines the layout of the window elements in the screen on the basis of the aspect ratio included in the loaded information by the CRT information loading unit 110, thus displaying the image whose size is most suitable for the screen size.

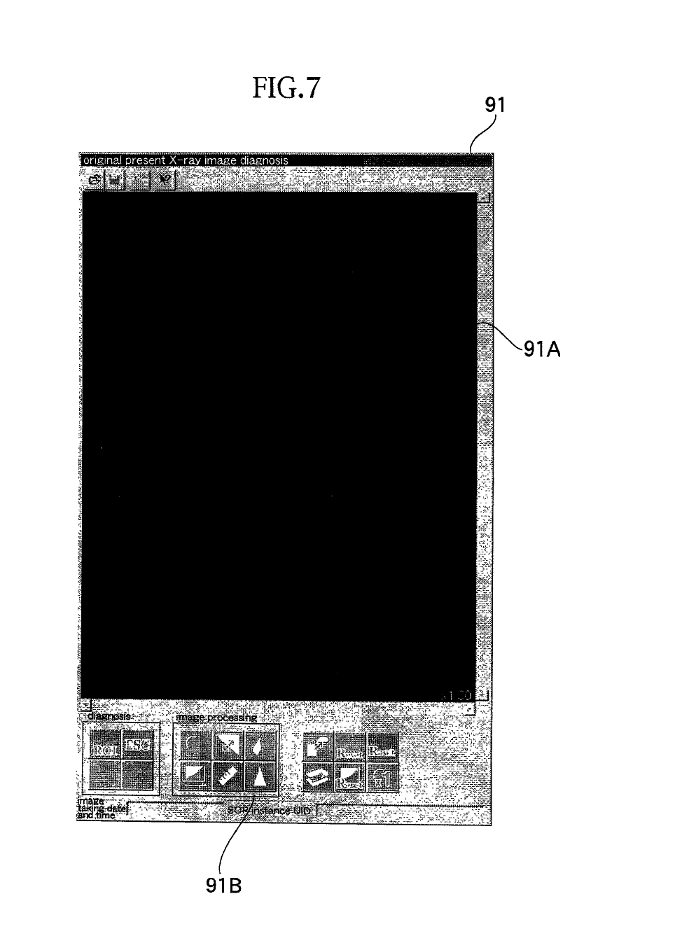

For example, when the screen of the CRT 60 has the horizontal side longer than the vertical side, as shown in FIG. 6A, it is possible to lay out the operation button area 91B in the left side of the image area 91A, but when the screen of the CRT 60 has the vertical side longer than the horizontal side, as shown in FIG. 7, it is possible to lay out the operation button area 91B in the bottom side of the image area 91A.

Next, a protection information setting/releasing unit 140 is explained.

The protection setting/releasing unit 140 corresponding to protection setting/releasing means sets or releases a protection information to/from the thumbnail image on the matrix image area shown in FIG. 2 so that the image corresponding to the protection information set thumbnail image is prevented from being deleted. The image protection information means for protecting the radiographic image or the produced temporally processed image, thus preventing the user from deleting the radiographic image or the produced temporally processed image in the data storage unit 20 by mistake. In this embodiment, it is possible for the user to confirm the protection information set state and the protection information release state of each thumbnail image.

When setting protection information to one thumbnail image, the user select with the input device 80 one desired thumbnail image on the matrix image area, which corresponds to the radiographic image or the temporally processed image.

For example, the input device 80 is a mouse, it is possible for the user to operate the mouse to click one desired thumbnail image on the matrix image area 50A, thus selecting the clicked thumbnail image. The user can select simultaneously only one thumbnail image corresponding to the radiographic image and only one thumbnail image corresponding to the temporally processed image.

After selecting one thumbnail image, when the user pushes down the diagnosis starting button 58 or another button corresponding to the diagnosis, the command for protecting the image corresponding to the selected thumbnail image is transmitted from the input device 80 to the protection setting/releasing unit 140.

The protection setting/releasing unit 140 sets the protection information of the image on the data storage unit 20, which corresponds to the selected thumbnail image, according to the transmitted command.

After reselecting the thumbnail image of the image to which the protection information is set, when the user pushes down the diagnosis starting button 58 or another button corresponding to the diagnosis, the command for releasing the protection information of the image corresponding to the selected thumbnail image is transmitted from the input device 80 to the protection setting/releasing unit 140.

The protection setting/releasing unit 140 releases the protection information of the image on the data storage unit 20, which corresponds to the selected thumbnail image, according to the transmitted command.

In a case where the image data is stored as a file in the data storage unit 20, the property information of the stored image file can be utilized for the protection information. Thus the property information is set to “read only” state so that it is possible to set the protection to “setting”. However, regardless of whether the image data is file format or not, providing a storage area capable of storing thereon the protection information corresponding to each image data in a data storing area of the data storage unit 20, such as the database makes it possible to generalize the setting of the protection information. In this embodiment, the latter system is employed.

As described above, when the user selects with the operation of the mouse one desired thumbnail image that the user intends to set the protection information or release it therefrom on the matrix image area 50A and pushes down the image protection button 58, the protection setting/releasing unit 140 processes the command which is transmitted from the input device 20 so as to rewrite the protection information of the corresponding image in the data storage unit 20 to “setting” or “releasing”.

That is, when the command for setting is transmitted, the protection setting/releasing unit 140 rewrites the protection information of the corresponding image in the data storage unit 20 to “setting”, and when the command for releasing is transmitted, the protection setting/releasing unit 140 rewrites the protection information of the corresponding image in the data storage unit 20 to “releasing”.

Next, a diagnosis completion information setting unit 150 is explained hereinafter.

The diagnosis completion information setting unit 150 corresponding to diagnosis completion information setting means sets a diagnosis completion information to the temporally processed image or/and the radiographic image. In other words, the diagnosis completion information setting unit 150 can store thereon whether the diagnosis of each temporally processed image and each radiographic image which is original thereto is already completed or not yet.

When setting the diagnosis completion information to one image, the user select with the input device 80 one desired image on the matrix image or the diagnosis image, which corresponds to the radiographic image or the temporally processed image. Concretely, the user operates the input device 80 to select the image (radiographic image or the temporally processed image) on the matrix image area or the diagnosis image. For example, the input device 80 is mouse, the user operates the mouse to click one desired image on the matrix image area or the diagnosis image. The user can select simultaneously only one image (radiographic image or temporally processed image).

After selecting one image (radiographic image or temporally processed image), when the user pushes down the diagnosis decision button 94C (referred to FIG. 5) or another button corresponding to the diagnosis decision, the command for diagnosis decision information is transmitted from the input device 80 to the diagnosis completion information setting unit 150.

The diagnosis completion information setting unit 150 processes the command that is transmitted from the input device 20 so as to rewrite the diagnosis completion information of the corresponding image in the data storage unit 20 to “diagnosis completed”.

Incidentally, the initial condition of the diagnosis completion information is set to “undiagnosed”.

In addition, in cases where the selected image is a temporally processed image, it is possible to collectively automatically set the selected temporally processed image and the original images to “diagnosis completed”, original images which are related to the temporally processed image in the data storage unit (database).

The diagnosis completion information setting unit 150 secures each area of each image data on which the diagnosis completion information with respect to each image data is capable of being stored so as to store on each secured area each state of “diagnosis completed” or “un-diagnosed” and the user's name (radiologist's name).

Next, a patient selecting unit 160 is explained hereinafter.

The patient selecting unit 160 corresponding to patient selecting means displays items of information of patients stored on the data storage unit 20 with the items of information tabulated so as to simultaneously select, according to the operation with the input device 80 by the user, a plurality of patients that the user intends to diagnose them on the tabulated image. The diagnosis image displaying unit 90 combines the stored temporally processed images in correspondence with each of the selected patients to the radiographic images which are the original images of the respective temporally processed images, thus sequentially displaying the combined images. As a result, the user sequentially reads the combined images so as to sequentially diagnose them.

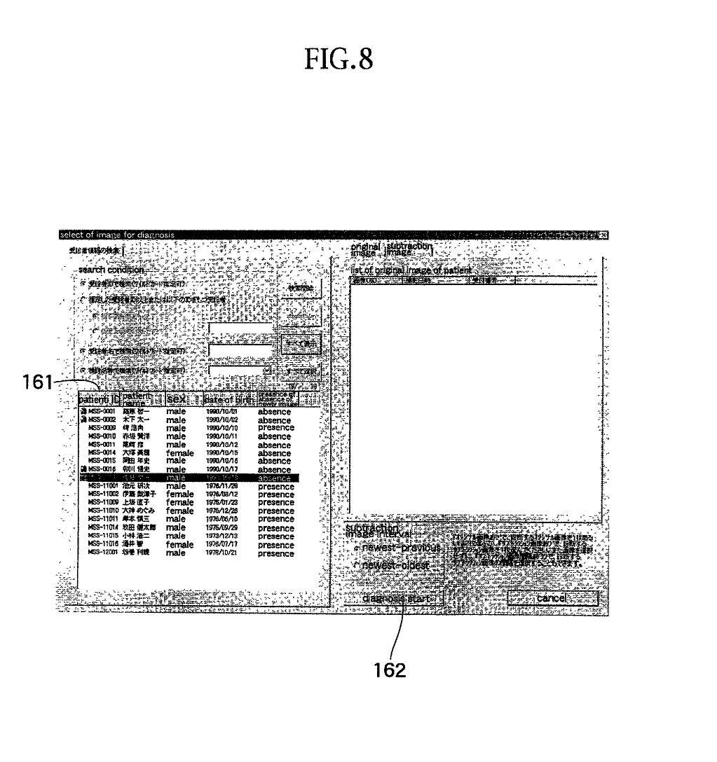

FIG. 8 shows an example of the tabulated image.

The tabulated image has a patient list area 161 on which each patient ID, each patient name, a number of stored images of each patient and so on are displayed so as to correspond to each other as patient items.

The user operates the input device 80 to select one or more patients (patient items). For example, the input device 80 includes a mouse and a keyboard, it is possible for the user to operate the mouse to click one or more patient items on the patient list area 161, or to click one or more patient items while pushing a key such as a shift key of the keyboard, thereby selecting one or more patients (patient items).

After selecting one or more patients, when the user pushes down a diagnosis starting button 162 or another button corresponding to the diagnosis, the command for displaying images corresponding to the selected patients is transmitted from the input device 80 to the diagnosis image displaying unit 90.

The diagnosis image displaying unit 90 displays the diagnosis images and the images (radiographic images, temporally processed images) corresponding to the selected patients on the screen of the CRT 60.

The diagnosis images are sequentially displayed in arbitrary order set by the system, for example, in order of selected patients, in ascending order or descending order of the selected patients IDs.

In addition, it is permitted to display a part of images of the patient without displaying all stored images of the patient.

For example, the temporally processed image produced from the newest radiographic image and the oldest radiographic image of the selected patient in the data storage unit 20, and the newest and oldest images may be displayed by the diagnosis image displaying unit 90, or the temporally processed image produced from the newest radiographic image and the radiographic image previous to the newest radiographic image of the selected patient in the data storage unit 20, the newest radiographic image and the radiographic image previous thereto may be displayed by the diagnosis image displaying unit 90.

The producing condition of the temporally processed images are not limited the above two cases, and able to be customized according to the command from the user (radiologist or the like).

When diagnosing next patient, the user pushes down a right allow button 94A in the control panel 94 shown in FIG. 5, or another button corresponding to a change of patient so that the diagnosis image display unit 90 changes over from the displayed images of the patient to new images of the next patient.

When diagnosing former patient, the user pushes down a left allow button 94B in the control panel 94 shown in FIG. 5, or another button corresponding to a change of patient so that the diagnosis image display unit 90 changes over from the displayed images of the patient to new images of the former patient.

Incidentally, the present invention is not limited to the above embodiment so that it is possible to carry out variety of modifications of the embodiment.

For example, in this embodiment, the layout of the window elements, the arrangement of the images, the input of the command and the selection of the images, which are executed by operating the pointing device such as mouse so as to click the window elements, are described on the basis of the window technique (graphical user interface), but the present invention is not limited to the technique.

In addition, it is possible to lay out the plural buttons in different arrangements, and to display different items of information on the buttons. For example, it is possible to display small-scaled image on the button so that the small-scaled image permits the user to easily imagine the function of the button, thereby assisting the user's operations.

Furthermore, in this embodiment, the arrangement of the radiographic images, the temporally processed images and the predetermined CAD (computer aided diagnosis) are explained, but the present invention may be applied to other images, or other CAD techniques.

While there has been described what is at present considered to be the preferred embodiment and modifications of the present invention, it will be understood that various modifications which are not described yet may be made therein, and it is intended to cover in the appended claims all such modifications as fall within the true spirit and scope of the invention.

What is claimed is:

1. An image display control system having a display unit, said image display control system comprising:
means for displaying a plurality of first thumbnail images of a plurality of radiographic images and a plurality of second thumbnail images of a plurality of temporally processed images on a screen of the display unit while the first and second thumbnail images are tabulated to form a matrix image,
said radiographic images being taken at different points of time, said temporally processed images being produced by combining the radiographic images;
means for selecting a plurality of the thumbnail images on the matrix image; and
means for sequentially displaying a plurality of images, said plurality of images corresponding to the selected thumbnail images.

2. An image display control system according to claim 1, wherein said sequentially displaying means displays, when the second thumbnail images are selected by the selecting means, the radiographic images which are original images of the temporally processed images corresponding to the selected thumbnail images.

3. An image display control system according to claim 1, wherein said sequentially displaying means displays the plurality of images in an order, and the order is permitted to be freely set.

4. An image display control system according to claim 1, further comprising, when an area on the matrix image which corresponds to a temporally processed image that is not produced yet, is selected, means for automatically producing the temporally processed image corresponding to the selected area.

5. An image display control system according to claim 1, further comprising means for setting protection information to at least one of the first and second thumbnail images on the matrix image, said protection information indicating that the image of the thumbnail image to which the protection information is set is prevented from being deleted, said protection information setting means being permitted to release the set protection information on the matrix image.

6. An image display control system according to claim 5, wherein a state such that whether the protection information is set to the thumbnail image or released therefrom is permitted to be confirmed on the matrix image.

7. An image display control system according to claim 1, further comprising means for setting a diagnosis completion information to at least one of the radiographic image and the temporally processed image, said diagnosis completion information indicating that a diagnosis of the corresponding image is already completed.

8. An image display control system according to claim 1, further comprising means for automatically adjusting a size of an window element according to a resolution of the screen, said window element being displayed on the screen.

9. An image display control system according to claim 1, further comprising means for automatically adjusting a layout in the screen according to an aspect ratio of the display unit.

10. An image display control system according to claim 1, further comprising means for storing thereon the temporally processed images and the radiographic images which are original images thereof while the temporally processed images and the radiographic images are related to each other.

11. An image display control system comprising:
means for storing thereon a plurality of items of information of patients, a plurality of radiographic images corresponding to the patients and a plurality of temporally processed images corresponding thereto;
means for displaying a list of the items of information of the patients so as to simultaneously select the items of information of the patients which are intended for diagnosis on the list; and
means for combining the stored temporally processed images and the radiographic images which are original images thereof, in correspondence with the selected patients, thus sequentially displaying the combined images.

12. An image display control system according to claim 11, wherein said sequentially displaying means displays the combined images in an order, and the order is permitted to be freely set.

13. An image display control method comprising the steps of:
preparing a display unit;
displaying a plurality of first thumbnail images of a plurality of radiographic images and a plurality of second thumbnail images of a plurality of temporally processed images on a screen of a display unit while the first and second thumbnail images are tabulated to form a matrix image,
said radiographic images being taken at different points of time, said temporally processed images being produced by combining the radiographic images;
selecting a plurality of the thumbnail images on the matrix image; and
sequentially displaying a plurality of images, said plurality of images corresponding to the selected thumbnail images.

14. An image display control method comprising the steps of:
storing on a data storage unit a plurality of items of information of patients, a plurality of radiographic images corresponding to the patients and a plurality of temporally processed images corresponding thereto;
displaying a list of the items of information of the patients so as to simultaneously select the items of information of the patients which are intended for diagnosis on the list; and
combining the stored temporally processed images and the radiographic images which are original images thereof, in correspondence with the selected patients, thus sequentially displaying the combined images.
